(19) United States
(12) Patent Application Publication
(10) Pub. No.: US 2023/0018531 A1
(43) Pub. Date: Jan. 19, 2023

(54) UROFLOW MEASUREMENT DEVICE IMPLEMENTING LOAD CELL BASED WEIGHT MEASUREMENT USING RIGID SUPPORT PASSING THROUGH SPACER WITHIN FLEXIBLE DIAPHRAGM OF HOUSING

(71) Applicant: Stream Dx, Inc, South Salt Lake, UT (US)

(72) Inventors: Kent James Ogden, Murray, UT (US); Alvin Y. Le, Riverton, UT (US); William J. Anderl, Holladay, UT (US); Brian D. Holt, North Salt Lake, UT (US); Scott B. McClellan, Park City, UT (US)

(73) Assignee: Stream Dx, Inc, South Salt Lake, UT (US)

(21) Appl. No.: 17/861,002

(22) Filed: Jul. 8, 2022

Related U.S. Application Data
(60) Provisional application No. 63/220,067, filed on Jul. 9, 2021.

(51) Int. Cl. A61B 5/20

(52) U.S. Cl. CPC A61B 5/208; A61B 2562/0252; A61B 2562/12

(57) ABSTRACT

A device includes: housing having top surface, bottom surface, and at least one side surface connected between top surface and bottom surface; a load cell positioned within housing; flexible section positioned within at least one of bottom surface or top surface of housing; rigid section positioned within flexible section, rigid section having opening surrounded by rigid material; rigid support having first end, second end, and center section; wherein first end of rigid support is positioned within housing and coupled to load cell; and wherein center section of rigid support passes through opening in rigid section positioned within flexible section, wherein center section of rigid support contacts rigid material of rigid section surrounding opening, wherein second end of rigid support is outside of housing.

### CROSS-REFERENCE TO RELATED APPLICATIONS

This application claims priority to U.S. Provisional Patent Application Ser. No. 63/220,067 filed on Jul. 9, 2021 and entitled “UROFLOW MEASUREMENT DEVICE IMPLEMENTING LOAD CELL BASED WEIGHT MEASUREMENT USING RIGID SUPPORT PASSING THROUGH SPACER WITHIN FLEXIBLE DIAPHRAGM OF HOUSING”, the entirety of which is incorporated herein by reference.

The following are hereby incorporated by reference in their entirety: (1) U.S. Provisional Patent Application Ser. No. 62/509,397 filed on May 22, 2017, entitled “CAPACITIVE MEASUREMENT DEVICE WITH INTEGRATED ELECTRICAL AND MECHANICAL SHIELDING”; (2) International Application PCT/US2018/033974 filed on May 22, 2018 and titled “CAPACITIVE MEASUREMENT DEVICE WITH MINIMIZED SENSITIVITY TO MANUFACTURING VARIABILITY AND ENVIRONMENTAL CHANGES”; (3) U.S. patent application Ser. No. 16/616,406 filed on Nov. 22, 2019, entitled “MEASUREMENT DEVICE WITH MINIMIZED SENSITIVITY TO MANUFACTURING VARIABILITY AND ENVIRONMENTAL CHANGES”; and (4) U.S. patent application Ser. No. 14/884,591 entitled “CAPACITIVE MEASUREMENT DEVICE WITH INTEGRATED ELECTRICAL AND MECHANICAL SHIELDING” filed on Oct. 15, 2015 (hereinafter the '591 Application).

### BACKGROUND

Uroflowmetry is the measure of the volume of urine released from the body, the rate at which urine is voided, and the time it takes to complete a voiding event. The results of a uroflowmetry test can be very beneficial in evaluating the health of the urinary tract. This test and detailed bladder diaries can also be very valuable in diagnosing abnormal health conditions, such as lower urinary tract symptoms, benign prostatic hypertrophy, prostate cancer, bladder tumor, neurogenic bladder dysfunction, urinary incontinence, urinary blockage, urinary tract infection, over-active bladder, stress urinary incontinence, as well as other conditions. Traditionally, uroflowmetry tests are conducted at a medical facility, such as a hospital or clinic. Testing in an artificial clinical setting opposed to a natural setting such as the patient's home can have a significant impact on the patient's performance. In addition to the obvious disadvantages of inconvenience and patient compliance, one complication that often arises with in-clinic testing is that the patient will need to urinate while waiting for the test to be administered. This can result in premature voiding or abnormal voiding events, which skew or negate the value of the test and require the patient to return to the clinic multiple times to get accurate results. Recent research in the area have demonstrated the benefits of multiple voids over multiple days. Due to intra-patient voiding variability “30 uroflow tracings are necessary for an accurate measurement”. See https://www.auajournals.org/doi/pdf/10.1097/JU.0000000000001504 incorporated herein by reference in its entirety. Multiple voids over multiple days are not practical in the clinic setting, thus requiring a clinically accurate, reliable, durable device for at home use.

### SUMMARY

A device includes: a housing having a top surface, a bottom surface, and at least one side surface connected between the top surface and the bottom surface; a load cell positioned within the housing; a flexible section positioned within at least one of the bottom surface or the top surface of the housing; a rigid section positioned within the flexible section, the rigid section having an opening surrounded by rigid material; a rigid support having a first end, a second end, and a center section; wherein the first end of the rigid support is positioned within the housing and coupled to the load cell; and wherein the center section of the rigid support passes through the opening in the rigid section positioned within the flexible section, wherein the center section of the rigid support contacts the rigid material of the rigid section surrounding the opening, wherein the second end of the rigid support is outside of the housing.

A device includes: a housing having a top surface, a bottom surface, and at least one side surface connected between the top surface and the bottom surface; a load cell positioned within the housing; a flexible section positioned within at least one of the bottom surface or the top surface of the housing, the flexible section having an opening; a rigid support having a first end, a second end, and a center section; wherein the first end of the rigid support is positioned within the housing and coupled to the load cell; and wherein the center section of the rigid support passes through the opening in the flexible section, wherein the center section of the rigid support is in contact with the flexible section.

A method of manufacturing a device includes: positioning a load cell within a housing, the housing having a top surface, a bottom surface, and at least one side surface connected between the top surface and the bottom surface; positioning a flexible section within at least one of the bottom surface or the top surface of the housing, the flexible section having an opening; passing a rigid support through the opening in the flexible section, wherein a center section of the rigid support is in contact with the flexible section; positioning a first end of the rigid support within the housing; and coupling the first end of the rigid support to the load cell within the housing.

### DRAWINGS

Understanding that the drawings depict only exemplary embodiments and are not therefore to be considered limiting in scope, the exemplary embodiments will be described with additional specificity and detail through the use of the accompanying drawings, in which:

FIG. 1A is a top perspective view of an example of a urine measurement device, including a container portion (cup) and an electronics portion.

FIG. 1B is a side view of the electronics portion (base) and container portion (cup) assembly with some detail regarding internal components.

FIG. 1C is a cross section side view of the electronics portion (base) and container portion (cup) assembly with detail regarding both mechanical and electronic major components.

FIG. 1D is a perspective view of container walls that are non-curved (such as square/rectangular).

FIG. 2A is a cross section side view of the electronics portion (base) illustrating detail of both mechanical and electronic components comprising the electronics portion (base).

FIG. 2B is an exploded perspective view of the electronics portion (base) showing detail of the components facilitating force translation from the material placed into the container portion (cup) to the load cell.

FIG. 2C is a top view of the bottom surface of the electronics portion (base) illustrating detail of the mechanical components comprising the electronics portion (base).

FIG. 2D is a cross section view of a flexible section (such as a diaphragm) and a rigid section (such as a spacer) positioned within a bottom surface or a top surface of a housing (such as electronics portion (base)).

FIG. 3: A top view of the electronics portion (base) with other sensors capable of measuring various aspects of the fluid or substance being measured.

FIG. 4: Is a block diagram of an exemplary system enabling transmission of data from a substance measurement device to a remote server.

FIG. 5: Is a diagram including both measurement of urine data and fluid consumed data using a urine measurement device and a smart device.

FIG. 6: Is a diagram showing a urine measurement device (such as a derivative of urine measurement device) that is also capable of performing portions of a urine analysis test.

In accordance with common practice, the various described features are not drawn to scale but are drawn to emphasize specific features relevant to the exemplary embodiments. Like reference numbers and designations in the various drawings indicate like elements.

### DETAILED DESCRIPTION

In the following detailed description, reference is made to the accompanying drawings that form a part hereof, and in which is shown by way of illustration specific illustrative embodiments. However, it is to be understood that other embodiments may be utilized and that logical, mechanical, and electrical changes may be made. Furthermore, the method presented in the drawing figures and the specification is not to be construed as limiting the order in which the individual steps may be performed. The following detailed description is, therefore, not to be taken in a limiting sense.

An example device is an electronic scale with various features designed to optimize its use, accuracy, durability, reliability, and ease of disinfection for repeated use and cleaning. These features include various flexible diaphragms (such as diaphragm 212), spacers (such as spacer 210), a consumable container (such as container portion 102), a removable bottom plate (such as bottom metal plate 110), a communication module, a flexible outer sleeve and bright interpretable LEDs to communicate functional status to a user.

The present design of a flow meter is a device that measures the weight of a substance (a liquid) that enters a container over a specific period of time. A uroflow device tracks the flow and volume of urine into a container. The flow and voiding profile can be representative of normal and abnormal voiding events. The data can be used by qualified medical professionals to track the severity and change of voiding conditions. In-clinic uroflowmetry faces various problems that can arise from logistical issues of scheduling patients to use the device to physiological factors that can dramatically change the voiding performance. Peak flow rate in men is generally accepted as a good indicator of the potential for urinary obstruction. Research shows that due to intra-patient voiding variability (high flow on one void, low flow on the next) the peak flow can vary by as much as 50% from the true mean peak flow. Additional research has shown that to measure peak flow to within 10% of the true mean peak flow requires 30 voids. Thirty (30) voids are not possible in the clinic setting. Achieving 30 voids requires sending a device to a patient's home where they can void naturally over a period of days. A device that can be sent to a patient's home must be simple to use, clinically accurate, rugged to withstand repeated shipping and handling, and easy to clean and disinfect for reuse.

Medication Therapy Management (MTM) includes medical care provided by pharmacists whose aim is to optimize drug therapy and improve therapeutic outcomes for patients. MTM can be a significant challenge because patient compliance as well as changing medical conditions place an emphasis on continuously monitoring or tracking the patient's condition. This high degree of patient condition monitoring often requires a hospital or intensive care unit setting. However, technology advances and through leveraging smart-devices, at home MTM can offer significant health benefits, especially for recovering heart failure patients.

The 30-day readmission rate for heart failure patients results in enormous additional expenses. Upon release from a hospital after a heart failure incident, a patient's health and recovery is largely dependent on the amount of fluids they consume and the amount of urine they void. Accumulation of fluids or fluid retention is bad for a recovering heart failure patient as it places additional load on the heart, potentially requiring hospital readmission. An excess of fluid loss can lead to dehydration which also places unnecessary additional load on the heart. Proper management of the patient's fluid load can significantly improve the patient's condition and minimize 30-day readmission rates to the hospital.

Proper management of the patient's fluid load can result in improved patient outcomes. Successful MTM generally includes collection of at least three things: (1) tracking urine voided; (2) tracking fluid intake; and (3) measuring urine creatinine (and other) levels over a 24-hour period. It has been difficult previously to effectively collect these three things at home or otherwise remote from a medical facility. Aspects of the examples described herein enable the collection of these three things at home or otherwise remote from a medical facility.

Due to the controlled engagement of the container and the electronics portion (base), it is foreseeable to include the ability for substance measurement. In examples, a urine measurement device includes a container portion and an electronics portion that are configured to be connected together. While this description places primary focus on uroflowmetry, it is understood that the description herein can apply more broadly to a substance measurement and/or analysis device that can make measurements about a substance entering into the container portion. In examples, the substance is urine or another bodily fluid, though other liquids, fluids, and/or solids could also be measured.

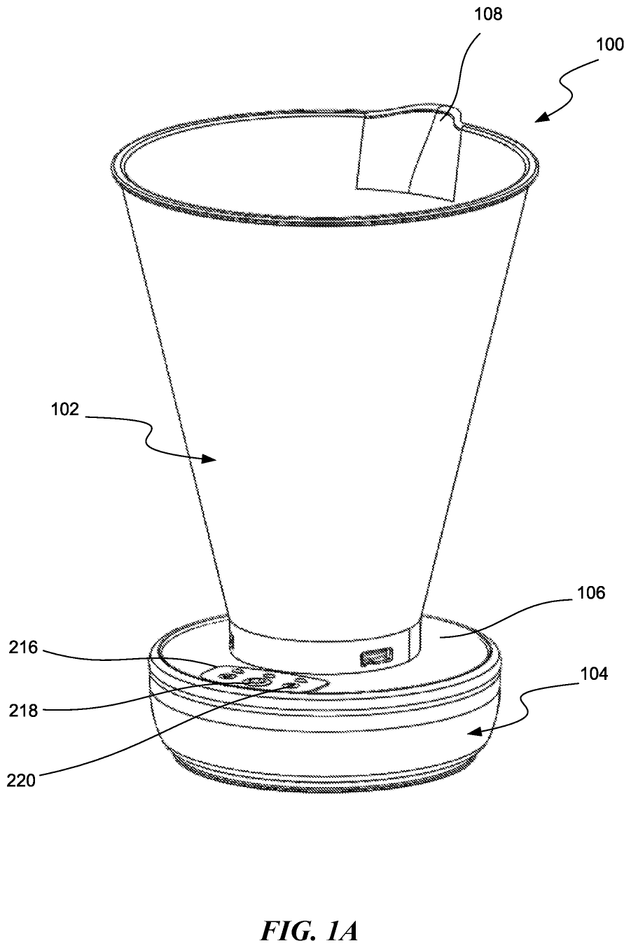

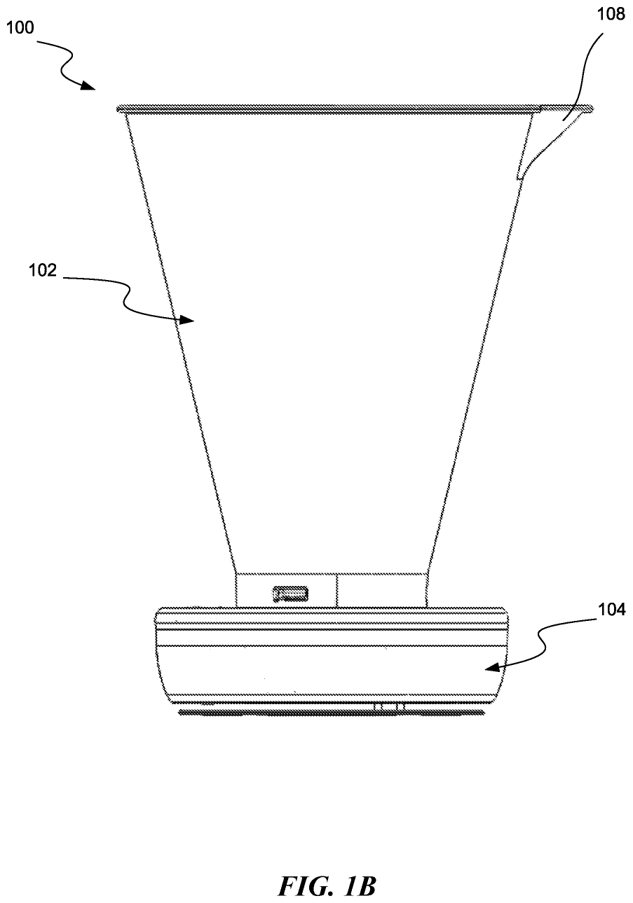

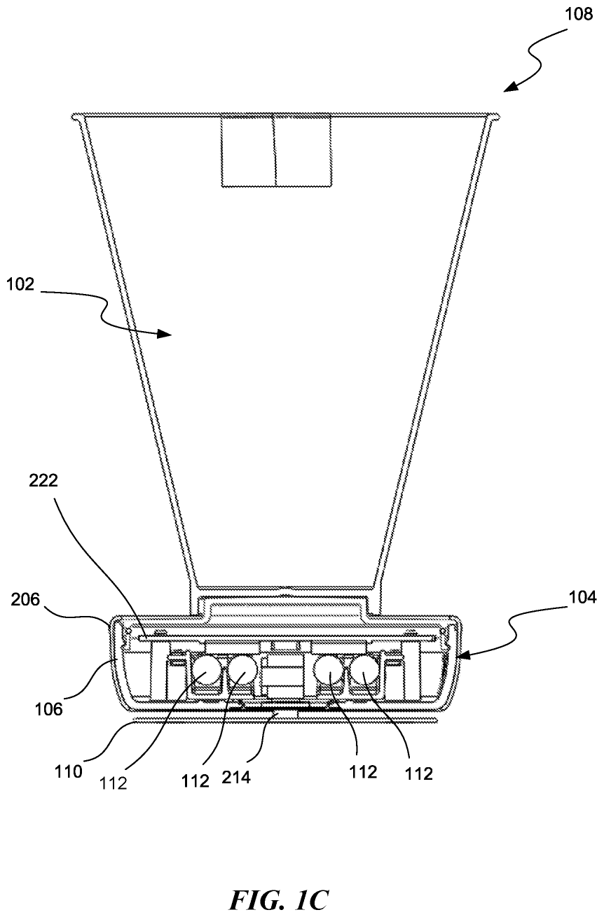

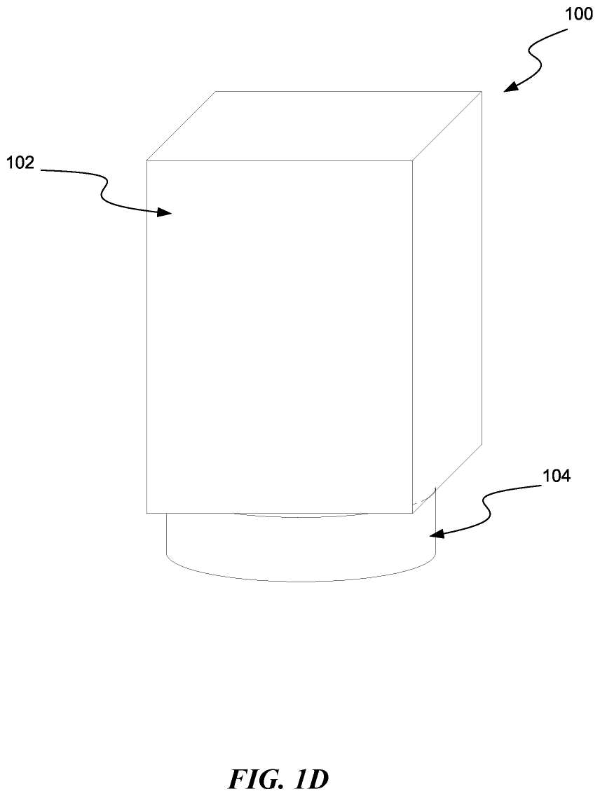

FIGS. 1A-1C show different views of an example of a urine measurement device 100 (or another fluid measurement device or another substance measurement device), including a container portion 102 (such as a cup or sleeve) and an electronics portion 104. FIG. 1A is a perspective view of the urine measurement device 100. FIG. 1B is a side view of the urine measurement device 100, displaying some detail of the electronics contained within the electronics portion 104 (base). FIG. 1C is a side view of the urine measurement device 100, which shows some detail and further detail when rotated 90 degrees. FIG. 1D is a perspective view of another exemplary embodiment of the container portion 102 of the urine measurement device 100 having a straight non-tapered side-wall with a square opening, square sides, and cube shape. The container portion could have rounded walls that are tapered (conical) or straight (cylindrical).

Examples of the container portion include a spout 108. In examples, a flexible outer sleeve (such as rubber sleeve 106) are included on the outside of the electronics portion 104. In examples, a flexible sleeve (such as rubber sleeve 106) is used to cover seams in the electronics portion 104 during patient use to make the seams fluid tight.

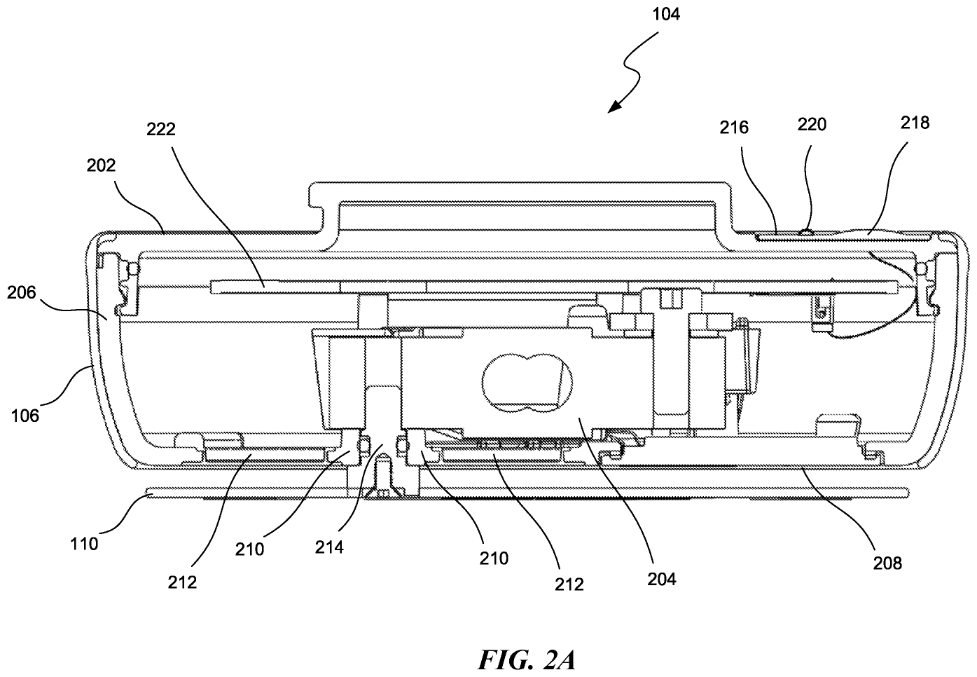

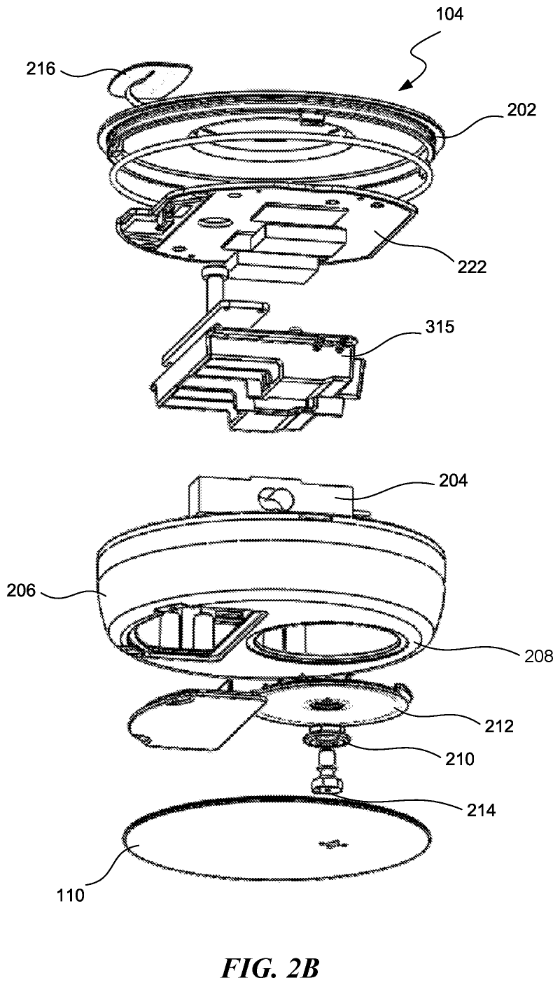

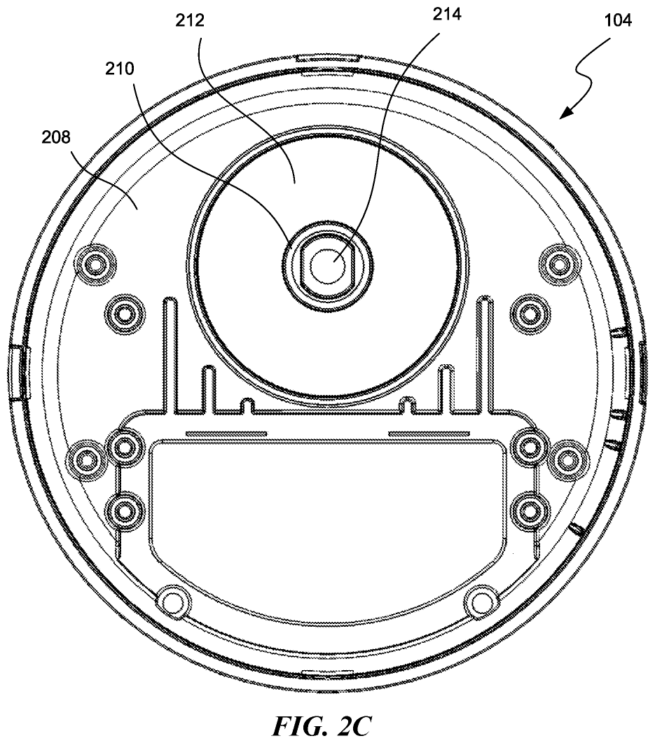

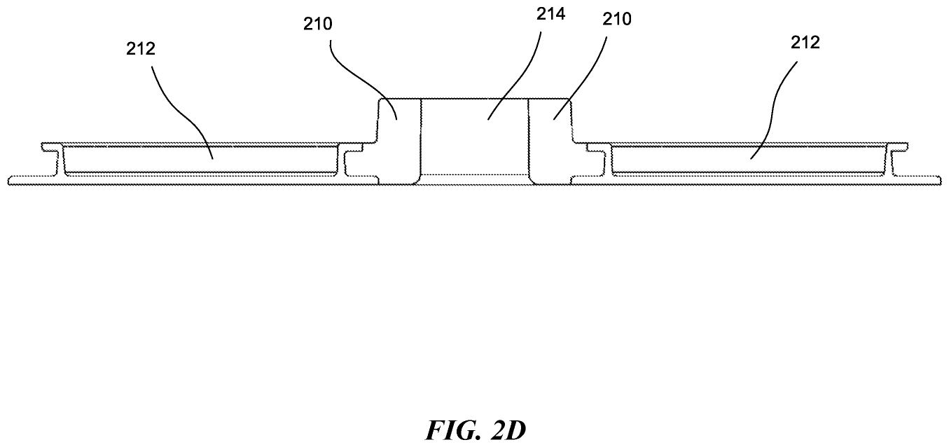

FIG. 2A is a cross section side view of the electronics portion 104 (base) illustrating detail of both mechanical and electronic components comprising the electronics portion 104 (base). FIG. 2B is an exploded perspective view of the electronics portion 104 (base) showing detail of the components facilitating force translation from the material placed into the container portion 102 (cup) to the at least one load cell 204. FIG. 2C is a top view of the bottom surface of the electronics portion 104 (base) illustrating detail of the mechanical components comprising the electronics portion 104 (base). FIG. 2D is a cross section view of a flexible section (such as a diaphragm 212) and a rigid section (such as a spacer 210) positioned within a bottom surface 208 or a top surface of a housing (such as electronics portion 104 (base)).

Aspects include a substance measurement device (such as urine measurement device 100) indicative of a level of the substance within the container portion 102; wherein the volume of the substance within the container portion 102 is determined based on the level of the substance within the container portion 102 and a known geometry of the container portion 102; and wherein the flow rate of the substance within the container portion 102 is determined by subtracting a first volume determined at a first time from a second volume determined at a second time to determine a difference between the second volume and the first volume and dividing the difference by an elapsed time between the first time and the second time. In examples, the substance is urine.

Aspects relate to a flexible section (such as diaphragm 212) and a rigid section (such as spacer 210). In examples, the housing (such as electronics portion 104) includes a top surface, a bottom surface 208, and at least one side surface connected between the top surface and the bottom surface 208. In examples, the flexible section (such as diaphragm 212) is positioned within at least one of the bottom surface 208 and the top surface of the housing (such as electronics portion 104). A diaphragm (such as diaphragm 212) made of flexible material (such as Thermoplastic Elastomer, Silicone Elastomer, Natural Rubber, etc.) which is meant to seal the inside of the device from any liquid contact.

Additionally, the diaphragm (such as diaphragm 212) is designed to move in such a way as to minimize any mechanical resistance to the motion of the at least one load cell 204 assembly. This is accomplished by attaching the rigid section (such as spacer 210, which can also be made of other rigid materials other than plastic) directly to the diaphragm (such as diaphragm 212). In examples, the rigid section (such as spacer 210) is positioned within the flexible section and the rigid section has an opening surrounded by rigid material. In examples, a rigid support (such as bottom bolt 214 or another bolt or support structure) is pushed through the rigid section (such as spacer 210) and secured to the at least one load cell 204. In examples, the rigid support (such as bottom bolt 214 or another bolt or support structure) has a first end, a second end, and a center section. In examples, the first end of the rigid support (such as bottom bolt 214 or another bolt or support structure) is positioned within the housing (such as electronics portion 104) and is coupled to the at least one load cell 204.

In examples, the center section of the rigid support (such as bottom bolt 214 or another bolt or support structure) passes through the opening in the rigid section (such as spacer 210) positioned within the flexible section (such as diaphragm 212). In examples, the center section of the rigid support (such as bottom bolt 214 or another bolt or support structure) contacts the rigid material of the rigid section surrounding the opening. In examples, the second end of the rigid support (such as bottom bolt 214 or another bolt or support structure) is outside of the housing (such as electronics portion 104). In examples, the flexible section (such as diaphragm 212) could be smaller in diameter than the bottom of the housing (such as electronics portion 104), or could make up the entire bottom surface 208 of the housing (such as electronics portion 104). In examples, diaphragm (such as diaphragm 212) thickness and specific geometry may vary. In examples, materials used for the diaphragm (such as diaphragm 212) are flexible, but may vary. In examples, the rigid section (such as spacer 210) is a solid piece, securely attached to the flexible section (such as diaphragm 212).

Aspects relate to a semi-permanent cup (such as container portion 102) attachment. Semi-permanent attachment points for the plastic cup (such as container portion 102) may use a twisting bayonet-mount style. This attachment point cause load placement of the plastic cup (such as container portion 102) and its contents to be consistent relative to the at least one load cell 204 across multiple measurements. Movement of the load during a measurement can significantly impact device accuracy. Examples may include different methods of attachment but should be focused on restricting the positioning of the fluid relative to the at least one load cell 204.

Aspects relate to a removable bottom plate (such as bottom metal plate 110). A plate (such as bottom metal plate 110) which creates the contact point with a mechanically static surface. The plate (such as bottom metal plate 110) is designed such that it can be removed between orders for cleaning. This enables a removable aspect of the design.

Aspects relate to a communication module that includes a combination of communication technologies such as Bluetooth, cellular and/or a combination of Bluetooth and/or cellular. In examples implementing Bluetooth, the Bluetooth allows the device to act as a data hub. The device is able to communicate with other paired devices such as an intake coaster, pH-meter, etc. In examples implementing Bluetooth, the device can connect to patient phone or other Bluetooth enabled device for data transfer or patient specific messages.

In examples implementing cellular, the patient data can be sent to a third party (such as Stream Dx) or directly to a physician. In examples implementing cellular, a third party (such as Stream Dx) can remotely control certain device functions via cellular communication. While cellular communication is described, these and similar features could also be implemented using Wi-Fi or other network connections.

In examples implementing a combination Bluetooth and cellular, the device can communicate with other paired devices and transmit data from all devices to Stream Dx or physicians. In examples, this may include unique data packaging strategies.

Aspects relate to a flexible outer sleeve (such as rubber sleeve 106). In examples, a flexible sleeve (such as rubber sleeve 106) used to cover seams in the device during patient use. This reduces the likelihood of the seams getting dirty and helps make the cleaning process between patients easier. It also increases friction for improved grip for elderly or neurologically impaired patients. In examples, the flexible outer sleeve is removable for cleaning.

Aspects relate to a bottom port for batteries (such as batteries 112). Examples implement a battery placement strategy in which rechargeable batteries are placed laterally on both sides of the load cell via a port on the bottom of the device.

Aspects relate to patient feedback. Examples implement the ability to give immediate feedback to patients regarding their use of the device (e.g., if the device is not level).

There may be importance and benefits relating to the following (among other things): (1) the way time is stored; (2) the order the data file is created (e.g, time, volume, accelerometer value, repeat); (3) the order and type of Information stored in the file header; (4) waking up the device periodically and taking a weight and level measurement to confirm that the device is tared; (5) taking measure(s) to prevent the bottom metal plate from rotating (keyed, or slotted, or alignment hole, etc.); (6) battery enclosure and base battery cover (how they function and maintain the battery connectivity).

In examples, the urine measurement device 100 is a device for use by men and/or women while voiding/urinating. In examples, funnels and/or other attachments may be placed on top of the container portion 102 and may have different shapes for male and female users. In examples, the funnel and/or geometry of the container portion 102 aid in minimizing splash back from urine or other substance that enters into the container portion 102. In examples, the device can be supported by a female urine collection device (commonly called a “Hat”) so that the user can be seated on the toilet during the measurement. In examples, the hand-held nature of the urine measurement device 100 introduces potential issues such as tilting and/or shaking of the urine measurement device 100 that may cause measurement errors of flow rate, fluid height, volume, etc. Features of the urine measurement device 100 described herein mitigate the effects of and/or reduce the likelihood of occurrence of the tilting and/or shaking of the urine measurement device.

Measuring fluid flow and fluid volume (such as urine) in a portable environment has many potential sources of error, including significant deviation from the horizontal plane (device is out of level), too much movement/motion while holding the device (sloshing and splashing), mal-alignment or miss-installation of the sleeve, weak or low batteries/power supply, a mal-functioning or defective sensor, an out of spec calibration sensor, an inaccurate calibration or failure to calibrate within a known specified tolerance among other possible failure modes. In examples, a sensor complement and/or a plurality of sensors (accelerometers and/or angular acceleration sensors) is used to determine the orientation of a weight-based collection vessel of urine or other bodily fluids for the purposes measuring flow and/or volume to improve accuracy. In examples, visual (such as from membrane keypad 216), audible, haptic or any other identifier are used to indicate an unacceptable or near unacceptable condition of a measurement system (that the device is within an acceptable horizontal level to take accurate data) prior to use. The warning may be delivered to the user via an app that is connected to the measuring device. In examples, error conditions (describing the problem and/or faulty condition(s)) are stored to memory and/or in a data file to facilitate post processing and data quality assessment.

In examples, the electronics portion 104 includes a printed circuit board (PCB) 222 with a controller that implements at least some of the processing described herein. In other examples, a controller is included in other portions of the electronics portion 104. In examples, the controller is a programmable processor, such as a microcontroller, an application-specific integrated circuit (ASIC), a field-programmable gate array (FPGA), a field-programmable object array (FPOA), or a programmable logic device (PLD). The controller described above may include or function with software programs, firmware or other computer readable instructions for carrying out various methods, process tasks, calculations, and control functions, described herein. These instructions are typically stored on any appropriate computer readable medium used for storage of computer readable instructions or data structures. The computer readable medium can be implemented as any available media that can be accessed by a general purpose or special purpose computer or processor, or any programmable logic device. Suitable processor-readable media may include storage or memory media such as magnetic or optical media. For example, storage or memory media may include conventional hard disks, Compact Disk-Read Only Memory (CD-ROM), volatile or non-volatile media such as Random Access Memory (RAM) (including, but not limited to, Synchronous Dynamic Random Access Memory (SDRAM), Double Data Rate (DDR) RAM, RAMBUS Dynamic RAM (RDRAM), Static RAM (SRAM), etc.), Read Only Memory (ROM), Electrically Erasable Programmable ROM (EEPROM), and flash memory, etc. Suitable processor-readable media may also include transmission media such as electrical, electromagnetic, or digital signals, conveyed via a communication medium such as a network and/or a wireless link.

The container portion 102 can be various shapes and sizes and can include or not include bottom walls and/or top walls. In examples, the container portion 102 includes a curved side-wall having an interior surface, an exterior surface, and a bottom surface. It does not include any top wall. Accordingly, in some embodiments, the container portion 102 is referred to as a sleeve, without a top or bottom. In these embodiments, the container portion 102 is only able to contain urine, another fluid, and/or substance once it is physically attached to the electronics portion 104, where the top surface of the electronics portion 104 serves as a bottom wall for the container portion 102. In other examples, the container portion 102 includes a bottom wall having an interior surface and an exterior surface, such that the container portion can contain urine, another fluid, and/or other substance even when it is not physically attached to the electronics portion 104. In examples, the container portion 102 includes at least one coupling element (such as threads, bayonet mounts, friction fit, tabs, clips, and/or fasteners) complimentary to at least one coupling element on the electronics portion 104. While the container portion 102 is generally referred to as having a curved side-wall, it is understood that embodiments of the container portion 102 can have straight side-walls and have different geometries.

In examples, the container portion 102 interfaces with an electronics portion 104 including at least one load cell 204, though it is understood that other types of sensors, such as resistive, magnetic, optical (visible light based, infrared light based, laser based, machine vision based), and other type of mechanical (such as weight based, pressure based, float based), radio-wave (such as radar based), acoustic (such ultrasound or infrasound) and capacitive ladder sensors can also be used. In examples, the at least one load cell 204 (or other sensor or sensing device) is configured to measure at least one of: (1) a flow rate of the urine, other liquid, or other substance into the container portion 102; (2) a volume of the urine, other liquid, or other substance within the container portion 102.

In examples, the container portion 102 is disposable and can be used only once or a few times before being disposed of. In other embodiments, the container portion is reusable many times. In examples, the electronics portion 104 is not disposable and is intended to be reused multiple times.

In examples, the electronics portion 104 is divided into a top portion 202 and a bottom portion 206 that can be separated to access the electronics inside of the electronics portion 104. In exemplary embodiment, the top portion 202 is positioned onto the bottom portion 206 using tabs on the top portion 202 and a keyed insert into bottom portion 206. In examples, the electronics portion 104 includes a top surface that serves as the bottom surface of the container portion 102 when the container portion does not include a bottom wall. In examples, the at least one coupling element (such as threads, bayonet mounts, friction fit, tabs, clips, and/or fasteners) is complimentary to the at least one coupling element. In examples, the at least one coupling element and the at least one coupling element engage after rotating the container portion 102 onto the electronics portion 104. In examples, when the at least one coupling element engages with the at least one coupling element, a liquid tight seal is created between the top surface and the bottom of the container portion 102. In the example of the container portion 102 that incorporates a bottom wall there may be a sealing element (O-ring) when the at least one coupling element and the at least one coupling element engage after rotating the container portion 102 onto the electronics portion 104.

In examples, the electronics portion 104 includes at least one button 218 and/or at least one electronic indicator 220 (such as a membrane LED status indicator) in a membrane keypad 216. In examples, the at least one button 218 can be pressed by the user to indicate the beginning and/or end of a voiding event during which measurement occurs. For example, the user could press the at least one button 218 before urinating into the container portion 102. The push of the at least one button 218 at this time can trigger a new voiding event file to be created by the electronics components within the electronics portion 104 and/or for a counter/timer marker to be placed into a file by the electronics components within the electronics portion 104. In examples, the user could press the at least one button 218 after completion of the voiding event once urination is completed. The push of the at least one button 218 at this time can trigger a counter/timer market to be placed into the file by the electronics components within the electronics portion 104, for writing to the file to be terminated, and/or for the file to be closed. In examples, writing to the file can be terminated and/or the file closed by the electronics components within the electronics portion 104 after the flow rate is below a certain threshold for a certain amount of seconds.

In examples, during and after a voiding event, any significant movement may add an indeterminate error to the voiding volume. Certain conditions and/or patient age may exacerbate this “shaking” which may add an indeterminate error. It would be advantageous for the user to convey to the device that their voiding event is actually over. This could assist in minimizing the hand-held “shaking” error that can potentially skew results. Additionally, placing the device on a level surface after the void to allow the device to take a “final” measurement of the actual void in a non-hand-held configuration could be used to “adjust” the final measured volume captured. In examples, the ability for the user to communicate to the device that a voiding event is over (a button push, voice command, etc.). In examples, a post void, “final” calibration on a level surface prior to shut down can be performed.

In examples, the electronics portion 104 can be activated in other ways rather than pressing a button or switch, such as by: (1) sensing the presence of urine, other fluid, and/or other substance within the container portion 102; (2) motion detection; (3) a sensor configured to detect when someone is holding the device; and/or (4) detection of when the electronics portion 104 is coupled with a container portion 102.

In examples, the at least one electronic indicator 220 includes at least one of a visual, audible, and haptic alert. For example, at least one of a visual, audible, and haptic alert can occur when: (1) the container portion 102 is properly connected with the electronics portion 104 once the at least one coupling element and the at least one coupling element are properly engaged; (2) the urine measurement device 100 and/or electronics portion 104 are functioning properly (such as to confirm operation after the urine measurement device 100 and/or electronics portion 104 were dropped); and (3) that the urine measurement device 100 is fully functional (battery level is adequate, container portion 102 is operating correctly, electronics portion 104 is operating correctly, any electrical contact between the container portion 102 and the electronics portion 104 is acceptable.

In exemplary embodiment, the at least one electronic indicator 220 is used to indicate when the at least one button 218 has been pressed and/or when the measurement is in progress or completed. In examples, the electronics portion 104 provides spoken instructions and/or status updates to the user, such as indications that the “device is fully functional” or requests for the user to “insert a sleeve”. In examples, audible beeps indicate operation and/or status updates to the user. In examples, the electronics portion 104 includes a more complex human machine interface (HMI) for user interaction with the urine measurement device 100. In examples, the HMI includes any combination of input and/or display devices, including for example light emitting diode (LED) indicators, Liquid Crystal Display (LCD) displays, e-ink displays, and/or touch screens, buttons, switches, dials, cameras, etc. In examples, haptic alerts include vibration.

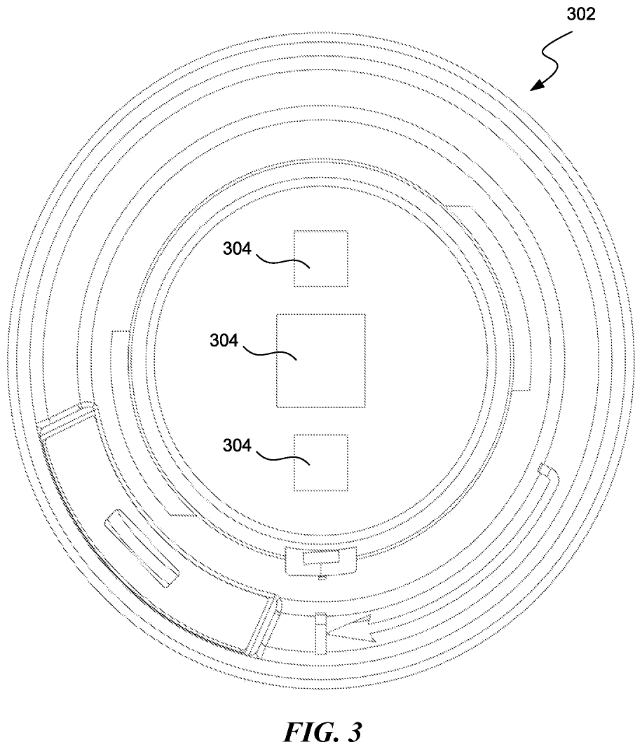

FIG. 3 show different views of another exemplary embodiment of the electronics portion 104 of the urine measurement device 100 having at least one sensor 304 on the top surface 142, referred to herein as electronics portion 302. FIG. 3 is a top view of the electronics portion 302. In examples, each of the at least one sensor 304 are particular sensors designed to sense various properties of the urine, other liquid, or other substance deposited into the container portion 102. In examples, the at least one sensor 304 senses proteins, dissolved solids, sugar levels, gravity, etc. of the urine, other liquid, or other substance deposited into the container portion 102. In examples, one of the at least one sensor 304 determines total dissolved solids (permittivity) in the urine, other liquid, or other substance. In examples, the at least one sensor 304 may be used to determine the PSA.

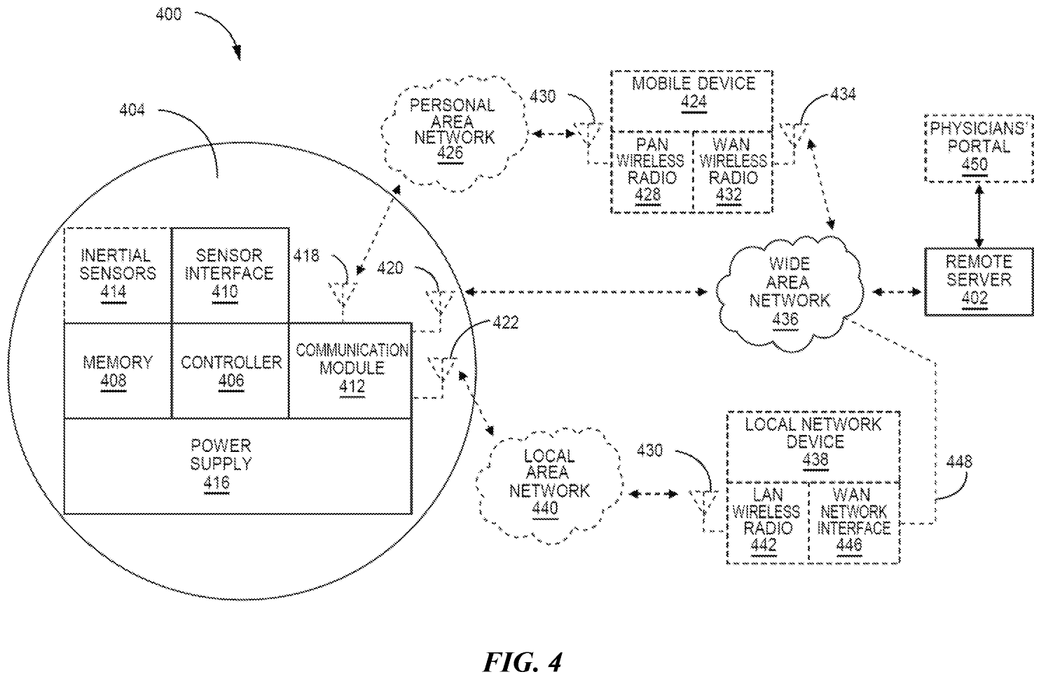

FIG. 4 is a block diagram of an example system 400 enabling transmission of data from a urine measurement device (such as urine measurement device 100) to a remote server 402. In examples, an electronics portion 404 (such as electronics portion 104 or electronics portion 302) of a urine measurement device is communicatively coupled with a remote server 402 in various ways. In examples, the electronics portion 404 includes a controller 406, memory 408, at least one sensor interface 410, at least one communication module 412, optional inertial sensors 414, and a power supply 416. In examples, the power supply provides power to the controller 406, memory 408, the at least one sensor interface 410, the at least one communication module 412, and the optional inertial sensors 414.

In examples, the controller 406 implements at least some of the processing described herein. In examples, the controller 406 is a programmable processor, such as a microcontroller, an application-specific integrated circuit (ASIC), a field-programmable gate array (FPGA), a field-programmable object array (FPOA), or a programmable logic device (PLD). The controller 406 described above may include or function with software programs, firmware or other computer readable instructions for carrying out various methods, process tasks, calculations, and control functions, described herein. These instructions are typically stored on any appropriate computer readable medium used for storage of computer readable instructions or data structures. The computer readable medium can be implemented as any available media that can be accessed by a general purpose or special purpose computer or processor, or any programmable logic device. Suitable processor-readable media may include storage or memory media such as magnetic or optical media. For example, storage or memory media may include conventional hard disks, Compact Disk-Read Only Memory (CD-ROM), volatile or non-volatile media such as Random Access Memory (RAM) (including, but not limited to, Synchronous Dynamic Random Access Memory (SDRAM), Double Data Rate (DDR) RAM, RAMBUS Dynamic RAM (RDRAM), Static RAM (SRAM), etc.), Read Only Memory (ROM), Electrically Erasable Programmable ROM (EEPROM), and flash memory, etc. Suitable processor-readable media may also include transmission media such as electrical, electromagnetic, or digital signals, conveyed via a communication medium such as a network and/or a wireless link.

In examples, at least one sensor interface 410 is configured to interface with at least one load cell 204 (or other sensor or sensing device) of the electronics portion and any additional at least one sensor 304 present in the urine measurement device. In examples, the controller 406 is configured to measure or calculate at least one of flow rate; urine, other fluid, and/or other volume based on the signals received at the at least one sensor interface 410 from the at least one load cell 204. In examples, the raw voltage value read from the sensors; the calculated flow rate; urine, other fluid, and/or volume is saved in the memory 408 or on another storage device. In examples, the raw voltage value read from the sensors, the calculated flow rate and/or urine, other fluid, and/or other volume is associated with a counter and/or time. In examples, the raw voltage value read from the sensors, the calculated flow rate and/or urine, other fluid, and/or other volume is transmitted to an external device using the at least one communication module 412. In examples, the at least one communication module 412 includes at least one processor as described herein, at least one radio, and/or at least one wired network interface. In other examples, the controller 406 is configured to take measurements of voltage that correspond to a weight applied to the load cell and through the at least one sensor interface 410 and to save the measurement in the memory 408 or on another storage device. In examples, the load cell measurements are associated with a counter and/or time. In examples, the load cell measurements or raw voltage values are transmitted to an external device using the at least one communication module 412. In examples data collected from the additional at least one sensor 304 is stored in the memory 408 or another storage device and/or transmitted to an external device using the at least one communication module 412.

In examples, the optional inertial sensors 414 are configured to provide inertial measurement data to the controller 406 that is used to compensate for any tilt of the container portion 102 during measurement of the flow rate and/or urine, other fluid, and/or volume. In examples, the optional inertial sensors include at least one accelerometer and/or at least one gyroscopes configured to measure tilt in particular directions and/or rotation around particular axes. In examples, the controller 406 compensates the data for any tilt and/or vertical perturbations detected by the optional inertial sensors 414 (such as an accelerometers detecting vertical error and/or gyroscopes detecting rotation/tilt) which could lead to false volumetric measurement if not mitigated. In other embodiments, the data from the inertial sensors is included with the data regarding the flow rate and/or urine, other fluid, and/or other substance height and/or volume that is provided to an external device for processing, such as the mobile device 424, local network device 438, and/or remote server 402 described herein and the external device performs the compensation of the data for any tilt and/or vertical perturbations detected by the optional inertial sensors 414 (such as an accelerometers detecting vertical error and/or gyroscopes detecting rotation/tilt) which could lead to false volumetric measurement if not mitigated.

In examples, data provided by the optional inertial sensors 414 is used to validate that the bottom of the container portion 102 is parallel to the ground, but is not used to actually compensate for the container portion 102 not being parallel to the ground. Instead, in examples when the data from the optional inertial sensors 414 indicates that the bottom of the container portion 102 is not parallel to the ground, an alert is provided to the user (such as through the indicator 148), so that the user can attempt to correct the issue. In examples, a visual and audible alarm can be used to alert the user that the device is out of horizontal level. The alarm could be related to other conditions of interest, low battery, excessive swirl (vibration), mal-alignment of the container component (sleeve), too much motion, sensor calibration issues, and/or sensor malfunction. In examples, the tilt indication comes through a visual indicator (such as tilt indicator light), an audible indicator (such as tilt indicator sound), and a haptic indicator (such as vibration). In addition, in examples when the data from the optional inertial sensors 414 indicates that the bottom of the container portion 102 is not parallel to the ground, a flag can be included in the measurement file to indicate that the device was not level during operation. In examples, the status of the operating conditions of interest are stored in data file either locally or remotely.

FIG. 4 shows three distinct data paths from the electronics portion 404 to the remote server 402, though additional paths are possible. In examples, the at least one communication module 412 includes a separate antenna for each data path, such as antenna 418, antenna 420, and antenna 422. In examples, the at least one communication module 412 communicates data to a mobile device 424 through a personal area network 426, such as a Bluetooth connection. In examples, the mobile device 424 includes a personal area network (PAN) wireless radio 428 having an antenna 430 and a wide area network (WAN) radio 432 having an antenna 434. In examples, at least one communication module 412 of the electronics portion 404 communicates signals via the antenna 418 across the personal area network 426 to the PAN wireless radio 428 of the mobile device 424 via the antenna 430. In examples, the mobile device 424 includes a processor that performs processing of the data received from the electronics portion 404. In examples, the mobile device 424 communicates data across a wide area network 436 to the remote server using WAN wireless radio 432 via antenna 434.

In examples, the at least one communication module 412 communicates data to the remote server 402 across the wide area network 436 using antenna 420. In examples, the at least one communication module 412 includes a cellular data modem and the wide area network 436 is implemented at least in part using a cellular data communication network.

In examples, the at least one communication module 412 communicates data to a local network device 438 across the local area network 440, such as a WiFi network, using antenna 422. In examples, the local network device 438 includes a local area network (LAN) wireless radio 442 having an antenna 444 and a wide area network (WAN) network interface 446 communicatively coupled to the wide area network 436 by a communication link 448. In examples, the at least one communication module 412 of the electronics portion 404 communicates signals via the antenna 418 across the local area network 440 to the LAN wireless radio 442 via the antenna 444. In examples, the local network device 438 includes a processor that performs processing of the data received form the electronics portion 404. In examples, the local network device 438 communicates data across a wide area network 436 via the communication link 448 to the remote server 402.

In examples, the communication link 448 is at least in part across a wired communication medium. In other examples, the communication link 448 is at least in part across a wireless communication medium. While wireless and/or wired communication elements are described herein, it is understood that other embodiments may include different types of communication in different areas of the system 400.

In examples, the electronics within the electronics portion 404, and specifically the controller 406, can be updated through over-the-air (OTA) firmware and/or software updates. In examples, the data collected at the remote server 402 can be accessed remotely by physicians, other health providers, and others using a physicians' portal 450. In examples, at least one of the controller 406, the mobile device 1124, the local network device 438, the remote server 402, the physicians' portal 450, and/or another device generates a voiding diary and/or other reports which can track various properties of urine over time including but not limited to volume, flow rate, glucose, blood, bacteria, color, odor, turbidity, specific gravity, pH, protein, ketones, urobilinogen, bilirubin, nitrites, leukocytes and creatinine.

In examples, data is captured and stored in on-board memory (SD card) and can be retrieved wirelessly, wired, or physically removing the storage device. In examples, data is sent to smart device (Android or iOS and stored) and then sent from the smart device to web-based database. In examples, data is sent to a web-based database via on-board cell module. In examples, the data transmitted includes at least one of date and time of events, device identification (ID), device health status, environmental conditions, environmental factors, sleeve identification (ID), calibration coefficients, etc. In examples, the data can be transmitted wirelessly or wired from the puck to a transmitting device, then from the transmitting device either wirelessly or wired to the Internet, such as a web portal or other server. In examples, the data can be transmitted in a data file including at least some data embedded in a header, footer, or any other location.

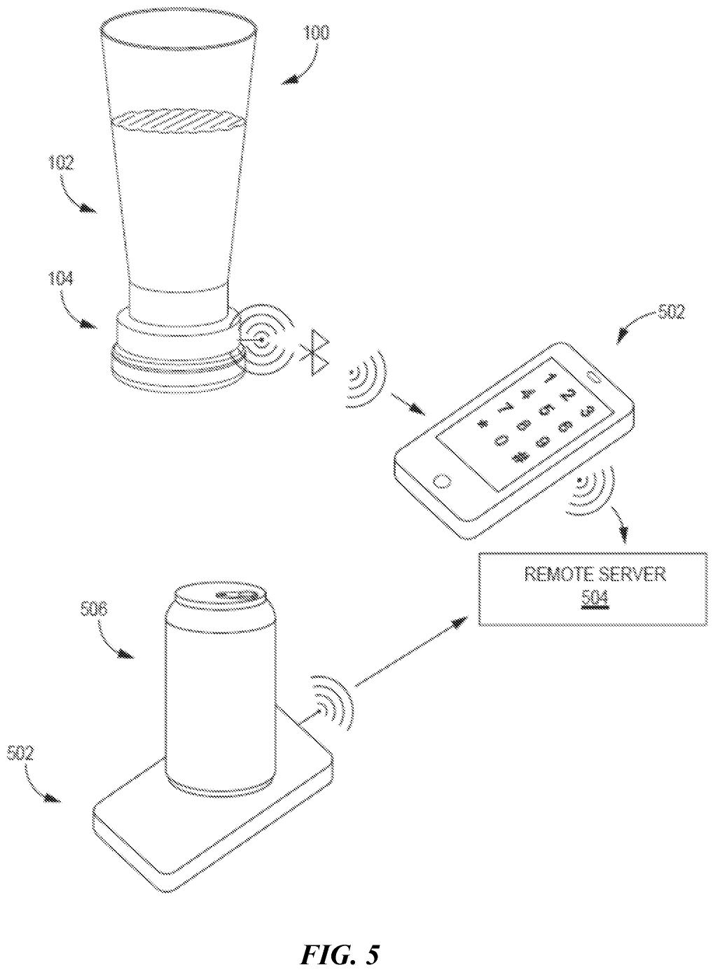

FIG. 5 is a diagram including both measurement of urine data and fluid consumed data using the urine measurement device 100 and a smart device 502. In examples, urine data is collected using the urine measurement device 100 (including the container portion and the electronics portion 104); which data is then transferred wirelessly to an app of a smart device 502 from the electronics portion 104; which data is then transmitted from the smart device 502 to a remote server 504, such as a web portal or other server through the Internet. This measuring of the voiding of urine is considered the “Outgo”. Another function of the app of the smart device (such as a smart phone or tablet) may include a fluid consumption or “Intake” app that tracks fluids consumed and sends data regarding the fluids consumed wirelessly to the same web portal or other server. In examples, the intake portion is accomplished by a smart device (such as a smart phone or tablet) taking the fluid weight directly by placing a container 506 with fluid directly on the screen of the smart device 502.

The twenty-four (24) hour voiding diary is valuable data for urologists treating patients with LUTS (lower urinary track symptoms). However, tracking fluid outgo is only part of the patient's condition. Tracking fluid intake would complete the diagnostic picture and could be used as feedback regarding MTM (medication therapy management) in the treatment of patients with heart disease and systemic fluid accumulation and/or patients at risk for pulmonary edema. The tracking of Intake fluids to be included with the voiding amounts and voiding frequencies to achieve a 24 hour intake/voiding diary. In examples, a pressure based measurement capability on smartphones is used to measure volume of fluids consumed as well as entering the type of fluid consumed. In examples, it also may be desirable to include not only weighing the fluid consumed and type of fluid consumed but weighing food to be consumed and the type of food consumed. Some smartphones (and other devices) include pressure sensitive displays. For example, Apple® iPhone® 6s and iPhone® 6s Plus included 3D Touch® which uses pressure sensitivity built into the device to sense how hard you press the display. The 3D Touch® display can not only sense multiple points of input, but can also sense how much pressure the user applies to the screen. By distinguishing different amounts of applied pressure applied by the user to the screen, the user can accomplish one action by pressing lightly and a different action by pressing harder. By pressing harder, the user produces pressure that allows the finger to press into the display on a microscopic level, allowing the user to interact with the display in three dimensions.

This pressure sensing capability of the screen on a smart device 502 (such as smartphones or other smart device, such as a tablet) allows the smart device 502 to be used to weigh things. A substance (such as a liquid) can be poured into a container placed on the display of the smartphone (or other smart device) and the pressure sensitive display on the smart device 502 (such as a smartphone or other smart device) can act as a scale, showing how much the material weighs. In examples, this pressure sensitive display on the smart device 502 (such as a smartphone or other smart device) is used to weigh fluid (and/or food or another substance) consumed by a patient, which can then be used to accurately track the amount of fluid (and/or food or another substance) consumed in a fluid intake diary (and/or food intake diary or another substance intake diary).

In examples, fluid (and/or food or another substance) intake is tracked with reference to medical or medication therapy. In examples, fluid amounts (and/or food or another substance) consumed and fluid types (and/or food types or another substance types) are tracked and stored on a smart device. In examples, the consumption data is stored on a remote server. While pressure sensitive smart devices are described herein as the means for measuring the fluid intake, it is understood separate weight or pressure based scales could also be used (such as an electronic “coaster” that transmits data via a wireless or wired connection).

Smart phones and other smart devices are ubiquitous. Enabling patients to easily and conveniently track their fluid intake with accuracy and precision using the pressure sensitivity built into the device that is already in their purse or pocket would be a huge step forward in making home or remote MTM a reality.

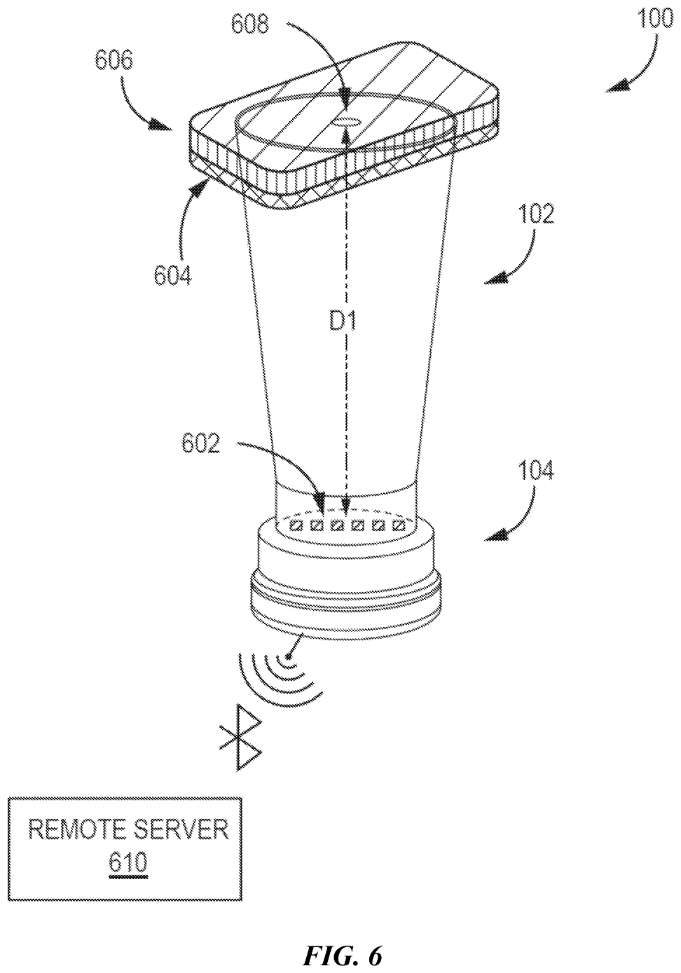

FIG. 6 is a diagram showing a urine measurement device 600 (such as a derivative of urine measurement device 100) that is also capable of performing portions of a urine analysis test. In examples, at least one of the container portion 102 and the top of the electronics portion 104 of the urine measurement device 600 are configured to receive at least one substance analysis reagent test strip 602 (such as urine analysis reagent test strips). In examples, the urine measurement device 100 further includes a lid portion 604. In examples, the container portion 102 and the lid portion 604 are light blocking (to limit light pollution from degrading the accuracy of reading the urine analysis reagent test strips with the smart device camera and flash). The lid portion 604 serves the purpose of providing a support structure for a smart phone 606 (or tablet or other smart device) with a camera 608 (or a stand-alone camera not part of a smart phone, tablet, or other smart device), blocking light into the container portion 102, and providing a constant distance D1 from the lens of the camera 608 to the at least one substance analysis reagent test strip 602, each of which aid in achieving an accurate reading of the urine analysis reagent test strips. In examples, the urine measurement device 100 is configured to transmit data to the remote server 610.

In examples, uroflowmetry is combined with urine analysis or urine analysis with uroflowmetry. Currently, uroflowmetry is a stand-alone test that measures the urine voided volume, flow profile, peak flow, average flow, time to peak flow, and other parameters of interest and importance to urologists. Urine analysis includes is a test where at least one substance analysis reagent test strip 602 is placed into urine and values from the urine constituents (such as, but not limited to, glucose, creatinine, ketones, blood, pH, protein, nitrites, leucocytes, urobilinogen, specific gravity, and bilirubin) are read off the at least one substance analysis reagent test strip 602 in a stand-along procedure using a separate urine analysis machine. As healthcare costs continue to rise and accessibility becomes more complicated, simple procedures and diagnostic tests will continue to move into the home or remote clinic settings. Currently, uroflow studies and urine analysis are two different procedures. In examples described herein, both uroflow and urine analysis can be completed during the same void, either simultaneously or sequentially.

Urine constituent analysis can occur using urine analysis reagent test strips exposed to urine and then read using a light (wavelength) reader. (http://www.healthcare.siemens.com/point-of-care/urinalysis/multistix-10-sg-reagent-strips/and http://usa.healthcare.siemens.com/point-of-care/urinalysis/). More recently there have been several App-based approaches to implementing urine analysis by reading urine analysis reagent test strips remotely using a smart device's camera feature (smartphone or tablet). These Apps can read partial or complete urine analysis reagent test strips, determining the concentration and/or presence of urine constituents such as but not limited to Leukocytes, Nitrites, Urobilinogen, Protein, pH, Blood, Specific Gravity, Ketone, Bilirubin, Glucose, and Creatinine (at least one sensor 304 in FIG. 3). These readings by a smart device can serve as a helpful indicator but have some basic limitations. The readings can be error prone due to variable ambient lighting conditions and distance D1 from the camera lens to the urine strip. Additionally, these urine analysis readings miss an important component in the overall diagnostic assessment of the LUTS patient, specifically collecting flow rate, volume, date and timing among other data.

While blood samples can provide good data regarding levels of various constituents in the body, urine is easier to analyze than blood. In examples, an app on a smart device is configured to take an image of at least one urine analysis reagent test strip that is on or in a portable, weight based urine flowmeter configured to measure the volume and flow rates of urine. While the description is described in the context of uroflowmetry and urine analysis, it is understood that flowmetry of other fluids and/or substances and analysis of the other fluid or substances can also be combined in similar ways. The urine analysis applies to a number of urine analysis constituents and other elements regarding a person's health and condition and is not limited in scope to any particular constituent. A number of potential urine constituents that are of particular interest in measuring during a urine analysis study are described at http://craigmedical.com/urinalysis_techs.htm, which is hereby incorporated by reference.

Creatinine is a waste product in your blood. It comes from protein in your diet and the normal breakdown of muscles of your body. Creatinine is removed from blood by the kidneys and then passes out of the body in your urine. If you have kidney disease, the level of creatinine in your blood increases. Blood (serum) and urine tests can check your creatinine levels. The tests are done to check how well your kidneys are working. A high serum or urine creatinine level can mean that your kidneys are not working well. A creatinine level may temporarily increase if you are dehydrated, have a low blood volume, eat a large amount of meat or take certain medications. Dietary supplemental creatinine can have the same effect.

Glucose is a simple sugar that circulates in the blood of animals as blood sugar. It is an important source of energy for cellular respiration. The glucose reagent panel is specific for glucose and no substance other than glucose is known to give a positive result. The reactivity of the glucose test decreases as the Specific Gravity of the urine increases. Reactivity may also vary with temperature. Small amounts of glucose may normally be excreted by the kidneys, these amounts are usually below the sensitivity range of this test but on occasion may produce a color between the ‘Negative’ and the 100/5 color block and may be interpreted by the observer as positive. Glycosuria is the condition of glucose in urine. Normally the filtered glucose is reabsorbed by the renal tubules and returned to the blood by carrier molecules. If blood glucose levels exceed renal threshold levels, the un-transported glucose will spill over into the urine. A common cause of high glucose levels is diabetes mellitus.

Ketones are alternative fuels for the body that are made when glucose is in short supply, such as overnight and during dieting and fasting. They are made in the liver from the breakdown of fats. Ketones may form in the blood and make their way into urine. Too many ketones can make individuals sick. The ketone test reacts with acetoacetic acid in urine. It does not react with acetone or beta-hydroxybutyric acid. Some high specific gravity/low pH urines may give reactions up to and including ‘Trace’. Normal urine specimens usually yield negative results with this reagent. False positive results (trace or less) may occur with high pigmented urine specimens or those containing large amounts of levodopa metabolites. Ketone bodies such as acetoacetic acid, beta-hydroxybutyric acid, and acetone can appear in urine in small amounts. These intermediate by-products are associated with the breakdown of fat. Common causes of high ketone levels include diabetes mellitus, starvation, and diarrhea.

Blood in urine can be indicative of medical conditions. The significance of the ‘Trace’ reaction may vary among patients, and clinical judgment is required for assessment in an individual case. Development of green spots (intact erythrocytes) or a green color (free hemoglobin/myoglobin) on the reagent area within 60 seconds indicates the need for further investigation. Blood is often found in the urine of menstruating females. This test is highly sensitive to hemoglobin and thus compliments the microscopic examination. This test is equally sensitive to myoglobin as to hemoglobin. The sensitivity of this test may be reduced in urines with high specific gravity. Captopril may cause decreased reactivity. False positives reactions can be caused by certain oxidizing contaminants such as hypochlorite—microbial peroxiclase associated with urinary ‘tract infection may also give a false positive reaction. Levels of ascorbic acid normally found in urine do not interfere with this test. Hemoglobinuria is the presence of hemoglobin in the urine. Common causes of blood in urine include hemolytic anemia, blood transfusion reactions, massive burns, and renal disease. Hematuria is the presence of intact erythrocytes and is usually pathological. Common causes of hematuria includes kidney stones, tumors, glomerulonephritis, physical trauma, urinary tract infection, and Prostatitis (the swelling and inflammation of the prostate gland).

The pH level of urine can be indicative of certain medical conditions. The pH test area measures pH values generally within 1 unit in the range of 5-8.5 visually and 5-9 instrumentally with 5 being very acidic and 8.5 being highly alkaline. Generally, urine pH results range from 5.5-7.5 in a bell curve type statistical distribution. Average for normal human urine is slightly acidic 6.0, however deviations from normal in any given sample are unremarkable and consistent, repeated readings are required in the top or bottom range to suggest an abnormality. High protein diets increase acidity. Vegetarian diets increase alkalinity. Bacterial infections also increase alkalinity producing a urine pH in the higher 7-8 range.

Protein in urine can be indicative of certain medical conditions. The reagent area is more sensitive to albumin than to globulins, hemoglobin, and mucoprotein. A ‘Negative’ result does not rule out the presence of other proteins. Normally no protein is detectable in urine by conventional methods, although a minute amount is excreted by the normal kidney. A color matching any block greater than ‘Trace’ indicates significant proteinuria. For urine of high specific gravity, the test area may most closely match the ‘Trace’ color block even though only normal concentrations of protein are present. Clinical judgment is needed to evaluate the significance of ‘Trace’ results. False positive results may be obtained with highly alkaline urines. Albumin is normally too large to pass through glomerulus tissue. Therefore, elevated results Indicate abnormal increased permeability of the glomerulus membrane. Non-pathological causes of protein in urine include pregnancy, physical exertion, and increased protein consumption. Pathological causes of protein in urine include glomerulonephritis bacterial toxins and chemical poisons.

High levels of nitrite in urine can be indicative of certain medical conditions. This test depends upon the conversion of nitrate (derived from the diet) to nitrite by the action of principally gram negative bacteria in the urine. The test is specific for nitrite and will not react with any other substance normally excreted in urine. Pink spots or pink edges should not be interpreted as a positive result. Any degree of uniform pink color development should be interpreted as a positive nitrite test suggesting the presence of 100000 or more organisms per ml, but color development is not proportional to the number of bacteria present. A negative result does not in itself prove that there is no significant bacteriuria. Negatives may occur when urinary tract infections are caused by organism which do not contain reductase to convert nitrate to nitrite; when urine has not been retained in the bladder long enough (4 hours or more) for reduction of nitrate to occur; or when dietary nitrate is absent, even if organisms containing reductase are present and the bladder incubation is ample. Sensitivity of the nitrite test is reduced for urines with a high specific gravity. High abnormal readings indicate the presence of bacteria. Causes of high levels of nitrite in urine include urinary tract infection.

Leucocytes (also referred to as white blood cells) are cells that circulate in blood, urine, and other body fluids and are involved in counteracting foreign substances and disease. Normal urine specimens generally yield negative results. Positive results of small (+) or greater are clinically significant. Individually observed ‘Trace’ results may be of questionable clinical significance. However, ‘Trace’ results observed repeatedly may be clinically significant. ‘Positive’ results may occasionally be found with random specimens from females due to contamination of the specimen by vaginal discharge. Elevated glucose concentrations or high specific gravity may cause decreased test results. The presence of leukocytes in urine is referred to as pyuria (pus in the urine). Causes of leucocytes in urine include urinary tract infection and Prostatitis (the swelling and inflammation of the prostate gland).

Urobilinogen is a colorless by-product of bilirubin reduction. It is formed in the intestines by bacterial action on bilirubin. About half of the urobilinogen formed is reabsorbed and taken up via the portal vein to the liver, enters circulation and is excreted by the kidney. This test area will detect urobilinogen in concentrations as low as 3 mIU/L (milli-international units per liter) in urine. The reagent area may react with substances known to interfere with Ehrlich's reagent, such as p-aminosalicylic acid and sulphonamides. Atypical color reactions may be obtained in the presence of high concentrations of p-aminobenzoic. False negative results may be obtained if formalin is present. Highly colored substances, such as azo dyes and riboflavin may mask color development on the reagent area. Strip reactivity increases with temperature. The optimum temperature is 22-26 degrees centigrade. The absence of urobilinogen cannot be determined with this test. Bile pigment derived from breakdown of hemoglobin. The majority of this substance is excreted in the stool, but small amounts are reabsorbed into the blood from the intestines and then excreted into the urine. Causes of urobilinogen in urine includes hemolytic anemia and liver diseases.

Specific gravity of urine is a measurement of the density of urine. It is the relative proportions of dissolved solids in relationship to the total volume of the specimen. It reflects how concentrated or diluted a sample may be. Water has a specific gravity of 1.000. Urine will always have a value greater than 1.000 depending upon the number of dissolved substances (salts, minerals, etc.) that may be present. Very dilute urine has a low specific gravity value and very concentrated urine has a high value. Specific gravity measures the ability of the kidneys to concentrate or dilute urine depending on fluctuating conditions. Normal range 1.005-1.030, average range 1.010-1.025. The specific gravity test permits the determination of urine specific gravity between 1.000 and 1.030. In general, it correlates within 0.005 with values obtained with the refractive index method. For increased accuracy, 0.005 may be added to readings from urine with pH equal to or greater than 6.5. Elevated specific gravity readings may be obtained in the presence of moderate quantities (1 7.5 g/L) of protein. Low specific gravity of is associated with conditions like diabetes insipidus, excessive water intake, diuretic use or chronic renal failure.

Bilirubin is a pigment formed in the liver by the breakdown of hemoglobin and excreted in bile. Normally no bilirubin is detected in urine by even the most sensitive methods. Even trace amounts of bilirubin are sufficiently abnormal to require further investigation. Atypical result colors may indicate bile pigment abnormalities and the urine specimen should be tested further by more quantitative laboratory means. Metabolites of drugs which give a color at low pH, such as Pyridium and Serenium may cause false positives. Ascorbic acid concentrations of 1.42 mIU/L (milli-international units per liter) or greater may cause false positives. Bilirubin comes from the breakdown of hemoglobin in red blood cells. The globin portion of hemoglobin is split off and the heme groups of hemoglobin are converted into the pigment bilirubin. Bilirubin is secreted in blood and carried to the liver where it is conjugated with glucuronic acid. Some is secreted in blood and some is excreted in the bile as bile pigments into the small intestines. Bilirubin in urine can be caused by liver disorders, cirrhosis, hepatitis, and obstruction of bile duct.

Capturing urine analysis data from a smart device photograph of a urine analysis reagent test strip requires three (3) things: (1) determining the “yellowness” of the urine to subtract that “variability” aspect out of the reading, (2) a constant focal length for the optical measurement, and (3) a controlled or known ambient lighting condition. Embodiments described herein describe how to control these variables facilitating a consistent and accurate interpretation of the urine analysis constituent values. In examples, the urine analysis reagent test strips are used during or immediately after the voiding event where urine flow and/or volume was measured. The data values (such as height, volume, and/or flow rate) from the voiding event at particular time and/or date can be wirelessly communicated or communicated with wires to a remote server.

In examples, at least one substance analysis position within the container portion 102 (including on top of the electronics portion 104) is configured to position at least one substance analysis reagent test strip 602 within the substance. In examples, the at least one substance analysis reagent test strip 602 is a urine analysis reagent strip that is used for analysis of at least one of: Leukocytes, Nitrites, Urobilinogen, Protein, pH, Blood, Specific Gravity, Ketone, Bilirubin, Glucose, and Creatinine. In examples, the lid portion 604 blocks ambient light pollution into the container portion 102 and allows the camera 608 (and flash) to take a photograph of the at least one substance analysis reagent test strip 602 within the container portion 102 while providing the following for repeatable and accurate analysis of the photograph of the at least one substance analysis reagent test strip 602: (1) a known focal length for the photograph and/or a known distance D1 between the camera 608 and the at least one substance analysis reagent test strip 602 within the container portion 102; and (2) known ambient lighting condition and/or a controlled ambient lighting condition. In examples, the urine measurement device 100 is configured to transmit the photograph to the remote server 610 from the electronics portion 104.

Sending data wirelessly or wired connection or saving data directly to an internal memory device involve sending additional information (date, time, device serial number, general device health parameters, calibration, among other data elements). Additional information helps identify and track various electronic data capturing devices and the electronic components contained therein (component lot numbers, board sources, assembly data, etc.); to facilitate device identification for any reason, potential recalls, component end of life, etc.

### EXAMPLES

Example 1 includes a device, comprising: a housing having a top surface, a bottom surface, and at least one side surface connected between the top surface and the bottom surface; a load cell positioned within the housing; a flexible section positioned within at least one of the bottom surface or the top surface of the housing; a rigid section positioned within the flexible section, the rigid section having an opening surrounded by rigid material; a rigid support having a first end, a second end, and a center section; wherein the first end of the rigid support is positioned within the housing and coupled to the load cell; and wherein the center section of the rigid support passes through the opening in the rigid section positioned within the flexible section, wherein the center section of the rigid support contacts the rigid material of the rigid section surrounding the opening, wherein the second end of the rigid support is outside of the housing.

Example 2 includes the device of Example 1, wherein the flexible section is positioned within the bottom surface of the housing.

Example 3 includes the device of Example 2, further comprising: a bottom plate coupled to the second end of the rigid support outside of the housing.

Example 4 includes the device of any of Examples 1-3, wherein the flexible section is positioned within the top surface of the housing.

Example 5 includes the device of Example 4, further comprising: a top plate coupled to the second end of the rigid support outside of the housing.

Example 6 includes the device of any of Examples 1-5, wherein the center section of the rigid support includes a flexible or rigid material to connect an interface between the center section with the rigid section positioned within the flexible section.

Example 7 includes a device, comprising: a housing having a top surface, a bottom surface, and at least one side surface connected between the top surface and the bottom surface; a load cell positioned within the housing; a flexible section positioned within at least one of the bottom surface or the top surface of the housing, the flexible section having an opening; a rigid support having a first end, a second end, and a center section; wherein the first end of the rigid support is positioned within the housing and coupled to the load cell; and wherein the center section of the rigid support passes through the opening in the flexible section, wherein the center section of the rigid support is in contact with the flexible section.

Example 8 includes the device of Example 7, wherein the second end of the rigid support is outside of the housing.

Example 9 includes the device of Example 8, wherein the flexible section is positioned within the bottom surface of the housing.

Example 10 includes the device of Example 9, further comprising: a bottom plate coupled to the second end of the rigid support outside of the housing.

Example 11 includes the device of any of Examples 8-10, wherein the flexible section is positioned within the top surface of the housing.

Example 12 includes the device of Example 11, further comprising: a top plate coupled to the second end of the rigid support outside of the housing.

Example 13 includes the device of any of Examples 7-12, wherein the center section of the rigid support includes a flexible or rigid material to connect an interface between the center section with the rigid section positioned within the flexible section.

Example 14 includes a method of manufacturing a device, comprising: positioning a load cell within a housing, the housing having a top surface, a bottom surface, and at least one side surface connected between the top surface and the bottom surface; positioning a flexible section within at least one of the bottom surface or the top surface of the housing, the flexible section having an opening; passing a rigid support through the opening in the flexible section, wherein a center section of the rigid support is in contact with the flexible section; positioning a first end of the rigid support within the housing; and coupling the first end of the rigid support to the load cell within the housing.

Example 15 includes the method of Example 14, wherein a second end of the rigid support is outside of the housing.

Example 16 includes the method of Example 15, wherein the flexible section is positioned within the bottom surface of the housing.

Example 17 includes the method of Example 16, further comprising: coupling a bottom plate to a second end of the rigid support outside of the housing.

Example 18 includes the method of any of Examples 15-17, wherein the flexible section is positioned within the top surface of the housing.

Example 19 includes the method of Example 18, further comprising: coupling a top plate to a second end of the rigid support outside of the housing.

Example 20 includes the device of any of Examples 1-19, wherein the center section of the rigid support includes a flexible or rigid material to connect an interface between the center section with the rigid section positioned within the flexible section.

Although specific embodiments have been illustrated and described herein, it will be appreciated by those of ordinary skill in the art that any arrangement, which is calculated to achieve the same purpose, may be substituted for the specific embodiments shown. Therefore, it is manifestly intended that this invention be limited only by the claims and the equivalents thereof.

What is claimed is:

1. A device, comprising:
a housing having a top surface, a bottom surface, and at least one side surface connected between the top surface and the bottom surface;
a load cell positioned within the housing;
a flexible section positioned within at least one of the bottom surface or the top surface of the housing;
a rigid section positioned within the flexible section, the rigid section having an opening surrounded by rigid material;
a rigid support having a first end, a second end, and a center section;
wherein the first end of the rigid support is positioned within the housing and coupled to the load cell; and
wherein the center section of the rigid support passes through the opening in the rigid section positioned within the flexible section, wherein the center section of the rigid support contacts the rigid material of the rigid section surrounding the opening, wherein the second end of the rigid support is outside of the housing.

2. The device of claim 1, wherein the flexible section is positioned within the bottom surface of the housing.

3. The device of claim 2, further comprising:
a bottom plate coupled to the second end of the rigid support outside of the housing.

4. The device of claim 1, wherein the flexible section is positioned within the top surface of the housing.

5. The device of claim 4, further comprising:
a top plate coupled to the second end of the rigid support outside of the housing.

6. The device of claim 1, wherein the center section of the rigid support includes a flexible or rigid material to connect an interface between the center section with the rigid section positioned within the flexible section.

7. A device, comprising:
a housing having a top surface, a bottom surface, and at least one side surface connected between the top surface and the bottom surface;
a load cell positioned within the housing;
a flexible section positioned within at least one of the bottom surface or the top surface of the housing, the flexible section having an opening;
a rigid support having a first end, a second end, and a center section;
wherein the first end of the rigid support is positioned within the housing and coupled to the load cell; and
wherein the center section of the rigid support passes through the opening in the flexible section, wherein the center section of the rigid support is in contact with the flexible section.

8. The device of claim 7, wherein the second end of the rigid support is outside of the housing.

9. The device of claim 8, wherein the flexible section is positioned within the bottom surface of the housing.

10. The device of claim 9, further comprising:
a bottom plate coupled to the second end of the rigid support outside of the housing.

11. The device of claim 8, wherein the flexible section is positioned within the top surface of the housing.

12. The device of claim 11, further comprising:
a top plate coupled to the second end of the rigid support outside of the housing.

13. The device of claim 7, wherein the center section of the rigid support includes a flexible or rigid material to connect an interface between the center section with the rigid section positioned within the flexible section.

14. A method of manufacturing a device, comprising:
positioning a load cell within a housing, the housing having a top surface, a bottom surface, and at least one side surface connected between the top surface and the bottom surface;
positioning a flexible section within at least one of the bottom surface or the top surface of the housing, the flexible section having an opening;
passing a rigid support through the opening in the flexible section, wherein a center section of the rigid support is in contact with the flexible section;
positioning a first end of the rigid support within the housing; and
coupling the first end of the rigid support to the load cell within the housing.

15. The method of claim 14, wherein a second end of the rigid support is outside of the housing.

16. The method of claim 15, wherein the flexible section is positioned within the bottom surface of the housing.

17. The method of claim 16, further comprising:
coupling a bottom plate to a second end of the rigid support outside of the housing.

18. The method of claim 15, wherein the flexible section is positioned within the top surface of the housing.

19. The method of claim 18, further comprising:
coupling a top plate to a second end of the rigid support outside of the housing.

20. The device of claim 1, wherein the center section of the rigid support includes a flexible or rigid material to connect an interface between the center section with the rigid section positioned within the flexible section.
